(19) United States
(12) Patent Application Publication
(10) Pub. No.: US 2016/0273027 A1
(43) Pub. Date: Sep. 22, 2016

(54) METHODS FOR DETECTING NUCLEIC ACIDS PROXIMITY

(71) Applicant: BIO-RAD LABORATORIES, INC., Hercules, CA (US)

(72) Inventors: Steven T. OKINO, San Carlos, CA (US); Man CHENG, Danville, CA (US)

(73) Assignee: Bio-Rad Laboratories, Inc., Hercules, CA (US)

(21) Appl. No.: 15/034,548

(22) PCT Filed: Nov. 21, 2014
(86) PCT No.: PCT/US14/66822

§ 371 Date: May 5, 2016

Related U.S. Application Data
(60) Provisional application No. 61/909,283, filed on Nov. 26, 2013.

(51) Int. Cl. C12Q 1/68

(52) U.S. Cl. CPC C12Q 1/6813

(57) ABSTRACT

The present invention provides methods for determining whether two or more nucleic acid molecules or two or more regions of a nucleic acid molecule in a sample are in close proximity to each other due to direct or indirect physical interactions.

### CROSS-REFERENCE TO RELATED PATENT APPLICATIONS

The present application claims benefit of priority to U.S. Provisional Patent Application No. 61/909,283, filed Nov. 26, 2013, which is incorporated by reference for all purposes.

### BACKGROUND OF THE INVENTION

Interactions between nucleic acid molecules and regions of nucleic acid molecules, either direct physical interactions between the nucleic acids or indirect interactions through complexes with other molecules, are involved in the regulation of cellular processes. For example, DNA looping is involved in many cellular processes, including transcription, replication, and recombination. Additionally, RNA interaction with genomic DNA is able to influence and regulate the transcription of DNA.

### BRIEF SUMMARY OF THE INVENTION

The present invention provides methods of determining whether two or more nucleic acid molecules or two or more regions of a nucleic acid molecule in a sample are in close proximity to each other due to direct or indirect physical interaction. In some embodiments, the method comprises:
providing a mixture of nucleic acids;
compartmentalizing the mixture into a sufficient number of compartments such that co-localization in a compartment of nucleic acid molecules or regions of a nucleic acid molecule due to close proximity can be distinguished from random co-localization; and
detecting the presence of two or more nucleic acid molecules or two or more regions of a nucleic acid molecule in the same compartment; thereby determining that the two or more nucleic acid molecules or the two or more regions of the nucleic acid molecule in the sample are in close proximity to each other.

In some embodiments, the providing step comprises providing the mixture of nucleic acids under conditions such that proteins remain bound to the nucleic acid molecules or regions of the nucleic acid molecule in the mixture.

In some embodiments, two or more nucleic acid molecules are detected. In some embodiments, two or more regions of a nucleic acid molecule are detected.

In some embodiments, the two or more nucleic acid molecules or the two or more regions of the nucleic acid molecule are in close proximity to each other due to direct interactions. In some embodiments, the two or more nucleic acid molecules or the two or more regions of the nucleic acid molecule are in close proximity to each other due to indirect interactions in a complex of molecules. In some embodiments, the two or more nucleic acid molecules or the two or more regions of the nucleic acid molecule are in close proximity to each other due to indirect interactions in a nucleic acid-protein complex.

In some embodiments, the nucleic acids are double-stranded. In some embodiments, the nucleic acids are single-stranded. In some embodiments, the nucleic acids are DNA. In some embodiments, the nucleic acids are RNA.

In some embodiments, the method comprises analyzing each compartment for the presence or absence of the two or more nucleic acid molecules or two or more regions of the nucleic acid molecule.

In some embodiments, the detecting step comprises amplifying the nucleic acid molecules or the regions of the nucleic acid molecule. In some embodiments, the amplifying step comprises PCR, quantitative PCR, or real-time PCR.

In some embodiments, the detecting step comprises nucleotide sequencing the nucleic acid molecules or the regions of the nucleic acid molecule.

In some embodiments, the detecting step comprises detecting one or more agents that hybridize to the nucleic acid molecules or to the regions of the nucleic acid molecule. In some embodiments, the one or more agents are fluorophores.

In some embodiments, the method comprises:
contacting the nucleic acids with at least two agents, wherein the first agent hybridizes to a first nucleic acid molecule or a first region of a nucleic acid molecule and wherein the second agent hybridizes to a second nucleic acid molecule or a second region of a nucleic acid molecule; and
detecting the presence of the first agent and the second agent; thereby determining that the two or more nucleic acid molecules or the two or more regions of the nucleic acid molecule in the sample are in close proximity to each other.

In some embodiments, the first agent and the second agent combine to produce a signal that is not generated in the absence of the first agent, the second agent, or both.

In some embodiments, the providing step comprises isolating the nucleic acids from the sample and wherein the isolating does not substantially disrupt direct or indirect interactions between nucleic acid molecules or between regions of nucleic acid molecules in the sample. In some embodiments, the isolated nucleic acids are resuspended in a solution. In some embodiments, the isolated nucleic acids are resuspended in a solution comprising one or more reagents for detecting the nucleic acid molecules or the regions of the nucleic acid molecule. In some embodiments, the one or more reagents are oligonucleotide probes.

In some embodiments, the sample is an extract from an animal, plant, bacterial, or viral source. In some embodiments, the sample comprises one or more cells. In some embodiments, the sample comprises an isolated cell nucleus.

In some embodiments, the providing step comprises disrupting or dissolving a cell membrane of one or more cells. In some embodiments, the providing step comprises permeabilizing a cell membrane of one or more cells.

In some embodiments, the providing step comprises nucleic acid shearing or nuclease digestion of the nucleic acids. In some embodiments, the providing step comprises purifying the nucleic acids from other components in the sample.

In some embodiments, the compartmentalizing step comprises diluting the mixture. In some embodiments, the diluting comprises sequentially diluting the mixture to generate a plurality of dilutions and compartmentalizing each of the plurality of dilutions into a plurality of compartments. In some embodiments, the droplets are surrounded by an immiscible carrier fluid. In some embodiments, the compartmentalizing step comprises partitioning the mixture into microcapsules.

### DEFINITIONS

Unless defined otherwise, technical and scientific terms used herein have the same meaning as commonly understood by a person of ordinary skill in the art. See, e.g., Lackie, DICTIONARY OF CELL AND MOLECULAR BIOLOGY, Elsevier (4^{th} ed. 2007); Sambrook et al., MOLECULAR CLONING, A LABORATORY MANUAL, Cold Spring Harbor Lab Press (Cold Spring Harbor, N.Y. 1989). The term “a” or “an” is intended to mean “one or more.” The term “comprise,” and variations thereof such as “comprises” and “comprising,” when preceding the recitation of a step or an element, are intended to mean that the addition of further steps or elements is optional and not excluded. Any methods, devices and materials similar or equivalent to those described herein can be used in the practice of this invention. The following definitions are provided to facilitate understanding of certain terms used frequently herein and are not meant to limit the scope of the present disclosure.

The terms “close proximity” or “in close proximity,” as used with reference to two or more nucleic acid molecules or two or more regions of a nucleic acid molecule, refers to two or more nucleic acid molecules or regions of a nucleic acid molecule that directly or indirectly physically associate with each other. In some embodiments, two or more nucleic acid molecules or regions of a nucleic acid molecule that are in close proximity to each other directly physically associate with each other, for example but not limited to, by base-pairing (e.g., canonical Watson-Crick base pairing), association of nucleic acids in a triple helix-like structure, hydrogen bonding, other covalent or non-covalent interaction, or a chemical interaction. In some embodiments, two or more nucleic acid molecules or regions of a nucleic acid molecule that are in close proximity to each other indirectly physically associate with each other, for example but not limited to, by associating through a larger complex of molecules that may contain one or more proteins and/or other non-nucleic acid molecules. In some embodiments, two or more nucleic acid molecules or regions of a nucleic acid molecule are in close proximity to each due to indirect interactions in a nucleic acid-protein complex.

The term “nucleic acid region” refers to a segment of sequence within a nucleic acid molecule. In some embodiments, a nucleic acid region is a region of sufficient length for specific hybridization to occur with another nucleic acid segment within a nucleic acid molecule or for binding to a non-nucleic acid component (e.g., a protein) in a complex. For example, in some embodiments a nucleic acid region is about 10-100 bp, about 20-500 bp, about 50-500 bp, about 100-10,000 bp, about 100-1000 bp, or about 1000-5000 bp, e.g., about 10, 15, 20, 25, 30, 40, 50, 60, 70, 80, 90, 100, 150, 200, 250, 300, 400, 500, 600, 700, 800, 900, 1000, 2000, 3000, 4000, 5000 bp). In some embodiments, length of nucleic acid in a nucleic acid region is a region of sufficient length to be amplified in a PCR reaction. For example, standard PCR reactions generally can amplify between about 35 to 5000 base pairs.

In some embodiments, nucleic acid regions are “separated” by an intervening sequence of nucleic acid. In some embodiments, the intervening sequence separating the nucleic acid regions is at least 50, 100, 200, 500, 1000, 5000, 10,000, 15,000, 20,000, 25,000, 30,000, 40,000, 50,000 or more base pairs long.

The terms “nucleic acid” and “polynucleotide” interchangeably refer to deoxyribonucleotide (DNA) or ribonucleotide (RNA) and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, and peptide nucleic acids (PNAs). In certain applications, the nucleic acid can be a polymer that includes multiple monomer types, e.g., both RNA and DNA subunits.

The term “compartmentalizing,” as used with reference to a sample or mixture, refers to separating the sample or mixture into a plurality of portions, or “compartments.” Compartments can be solid or liquid. In some embodiments, a compartment is a solid compartment, e.g., a microchannel. In some embodiments, a compartment is a fluid compartment, e.g., a droplet. In some embodiments, a fluid compartment (e.g., a droplet) is an aqueous droplet that is surrounded by an immiscible carrier fluid (e.g., oil).

The term “agent” and “detectable agent” interchangeably refer to a composition detectable by spectroscopic, photochemical, biochemical, immunochemical, chemical, or other physical means. For example, useful agents include fluorescent dyes, luminescent agents, radioisotopes (e.g., ³²P, ³H), electron-dense reagents, enzymes, biotin, digoxigenin, or haptens and proteins, nucleic acids, or other entities which may be made detectable, e.g, by incorporating a radiolabel into an oligonucleotide that binds to a target nucleic acid molecule or nucleic acid region.

The term “specifically binds to” or “specifically associates with,” as used with reference to an agent binding to or associating with a component of a complex with which a nucleic acid physically associates, refers to an agent that binds to the component in the complex with at least 2-fold greater affinity than to non-complexed components, e.g., at least 3-fold, 4-fold, 5-fold, 6-fold, 7-fold, 8-fold, 9-fold, 10-fold, 20-fold, 25-fold, 50-fold, or 100-fold or greater affinity.

### BRIEF DESCRIPTION OF THE DRAWINGS

FIG. 1. Schematic of detecting nucleic acid proximity in compartments. A method to determine if two nucleic acid regions (e.g., DNA) are in close proximity to each other is depicted. In Sample 1, DNA regions A and B are not proximal to each other and there is no interaction between them. In Sample 2, DNA regions A and B are in close proximity to each other because proteins that are associated with regions A and B interact directly. The sample (Sample 1 or Sample 2) is compartmentalized into a plurality of compartments (e.g., a number of compartments greater than the number of A and B molecules), and the presence of A and/or B is detected for the compartments. For Sample 1, DNA regions A and B are detected most often in separate compartments, indicating that DNA regions A and B do not interact in Sample 1. For Sample 2, DNA regions A and B are detected most often in the same compartment, indicating the DNA regions A and B are in close association in Sample 2.

### DETAILED DESCRIPTION OF THE INVENTION

### I. Introduction

Methods and kits for determining whether two or more nucleic acid molecules or two or more regions of a nucleic acid molecule in a sample are in close proximity to each other are provided. Without being bound to a particular theory, it is believed that in a sample (e.g., a liquid sample), nucleic acids that are in close proximity due to physical interaction (e.g., direct or indirect physical association) will co-segregate when the sample (e.g., the liquid sample) is compartmentalized. Thus, nucleic acids that are in close proximity to each other will be found in the same compartment more often than nucleic acids that are not in close proximity to each other. By compartmentalizing the sample (e.g., the liquid sample) into a number of compartments and analyzing the compartments for the presence of the nucleic acids, valuable information about complex nucleic acid structures and interactions can be provided. For example, the methods, compositions, and kits described herein can be used for the identification of RNA, DNA, or chromatin molecules that interact with other RNA, DNA, or chromatin molecules and/or for the identification of RNA, DNA, or chromatin regions that interact with one another in an intramolecular interaction (i.e., looping).

### II. Detecting Nucleic Acid Proximity

In one aspect, methods of determining whether two or more nucleic acid molecules or two or more regions of a nucleic acid molecule in a sample are in close proximity to each other, due to direct or indirect physical interaction, are provided. In some embodiments, methods of determining whether two or more separate nucleic acid molecules in a sample are in close proximity due to direct or indirect physical interactions are provided. In some embodiments, methods of determining whether two or more separated regions of a single nucleic acid molecule in a sample are in close proximity due to direct or indirect physical interactions are provided. In some embodiments, the method comprises:
providing a mixture of nucleic acids;
compartmentalizing the mixture into a sufficient number of compartments such that co-localization of nucleic acid molecules in a compartment due to close proximity can be distinguished from random co-localization; and
detecting the presence of two or more nucleic acid molecules or two or more regions of a nucleic acid molecule in the same compartment; thereby determining that the two or more nucleic acid molecules or between the two or more regions of the nucleic acid molecule in the sample are in close proximity to each other.

In some embodiments, the method comprises analyzing each compartment for the presence or absence of the two or more nucleic acid molecules or two or more regions of the nucleic acid molecule and quantifying the number of compartments that are positive for the presence of each of the two or more nucleic acid molecules or two or more regions of the nucleic acid molecule. In some embodiments, the method comprises determining whether the number of compartments that are positive for the presence of each of the two or more nucleic acid molecules or two or more regions of the nucleic acid molecule exceeds the number of positive compartments that would be expected due to random co-localization of the nucleic acid molecules or regions of the nucleic acid molecule.

In some embodiments, close proximity due to direct physical interactions are detected. Direct interactions between nucleic acids include, for example, physical interactions such as base-pairing (e.g., canonical Watson-Crick base pairing), association of nucleic acids in a triple helix-like structure, hydrogen bonding, other covalent or non-covalent interactions, and chemical interactions.

In some embodiments, close proximity due to indirect physical interactions are detected. In indirect interactions between nucleic acids, two or more nucleic acid molecules or regions of a nucleic acid molecule are part of a larger complex of molecules that may contain proteins and/or other non-nucleic acid molecules. The nucleic acid molecules or regions of a nucleic acid molecule may or may not be in physical contact with each other. Indirect physical interactions include, for example, nucleic acid-protein complexes. In some embodiments, the nucleic acid-protein complex is a complex that is involved in regulation of nucleic acid transcription, replication, repair, recombination, or processing (e.g., a transcription initiation complex, an mRNA splicing complex, or an RNA-induced silencing complex). In some embodiments, wherein nucleic acids are in close proximity due to interactions via a nucleic acid-protein complex, the protein is a protein that interacts with a nucleic acid by a DNA- or RNA-binding domain (e.g., a transcription factor or an enzyme that modifies a nucleic acid at specific sites). In some embodiments, the protein is not a histone protein. In some embodiments, a nucleic acid-protein complex comprises chromatin.

In some embodiments, double-stranded nucleic acids in close proximity to each other are detected. In some embodiments, single-stranded nucleic acids in close proximity to each other are detected. In some embodiments, a double-stranded nucleic acid and a single-stranded nucleic acid in close proximity to each other are detected. In some embodiments, two or more DNA molecules (e.g., genomic DNA or cDNA) or two or more separated regions of a DNA molecule (e.g., genomic DNA or cDNA) in close proximity to each other due to direct physical interaction or indirect physical interaction (e.g., interaction of the two or more DNA molecules in a complex with a protein) are detected. In some embodiments, two or more RNA molecules (e.g., coding RNA (mRNA) or non-coding RNA, e.g., microRNA (miRNA), small interfering RNA (siRNA), or long non-coding RNA) or two or more separated regions of an RNA molecule (e.g., coding RNA or non-coding RNA) in close proximity to each other due to direct physical interaction or indirect physical interaction (e.g., interaction of the two or more RNA molecules in a complex with a protein) are detected. In some embodiments, DNA (e.g., genomic DNA) and RNA (e.g., mRNA) in close proximity to each other due to direct physical interaction or indirect physical interaction (e.g., interaction of the DNA and RNA molecules in a complex with a protein) are detected. In some embodiments, the sequences of the two or more nucleic acid molecules or two or more regions of a nucleic acid molecule are not identical or substantially identical.

### Samples

The methods described herein can be used to detect nucleic acid proximity due to direct or indirect physical interaction in any type of sample. In some embodiments, the sample is a biological sample. Biological samples can be obtained from any biological organism, e.g., an animal, plant, fungus, bacterial, or any other organism. In some embodiments, the biological sample is from an animal, e.g., a mammal (e.g., a human or a non-human primate, a cow, horse, pig, sheep, cat, dog, mouse, or rat), a bird (e.g., chicken), or a fish. In some embodiments, a sample for which nucleic acid interactions can be detected is from an animal, plant, bacterial, or viral source.

A biological sample can be any tissue or bodily fluid obtained from a biological organism, e.g., blood, a blood fraction, or a blood product (e.g., serum, plasma, platelets, red blood cells, and the like), sputum or saliva, tissue (e.g., kidney, lung, liver, heart, brain, nervous tissue, thyroid, eye, skeletal muscle, cartilage, or bone tissue), cultured cells, stool, urine, etc. In some embodiments, the sample comprises one or more cells. In some embodiments, the cells are animal cells, including but not limited to, human, or non-human, mammalian cells. Non-human mammalian cells include but are not limited to, primate cells, mouse cells, rat cells, porcine cells, and bovine cells. In some embodiments, the cells are plant or fungal (including but not limited to yeast) cells. Cells can be, for example, cultured primary cells, immortalized culture cells, or cells from a biopsy or tissue sample, optionally cultured and stimulated to divide before assayed.

In some embodiments, the sample comprises an isolated cell nucleus. Methods of isolating cell nuclei are known in the art. See, e.g., Marzluff, W. F., and Huang, R. C. C., “Transcription of RNA in Isolated Nuclei,” in Transcription and Translation: A Practical Approach, Hames B. D. and Higgens, S. J. (Eds.) pp 89-129 (IRL Press, Oxford, U K, 1984); Greenberg, M. E., and Bender, T. P., Identification of Newly Transcribed RNA, in Current Protocols in Molecular Biology, Ausubel, F. M., et al. (Eds.) pp. 4.10.1-4.10.11 (John Wiley and Sons, New York, 1997); and Farrell, Jr., R. E., Analysis of Nuclear RNA, in RNA Methodologies: A Laboratory Guide for Isolation and Characterization, Farrell, Jr., R. E. (Ed.) pp. 406-437 (Academic Press, San Diego, 1998).

In some embodiments, nucleic acid molecules or regions of nucleic acid molecules, or sub-fractions comprising target nucleic acid molecules or regions of nucleic acid molecules, are extracted or isolated from a sample (e.g., a biological sample). In some embodiments, the extraction or isolation of nucleic acids (e.g., nucleic acid molecules or regions of nucleic acid molecules) does not substantially disrupt direct or indirect interactions between nucleic acid molecules or between regions of nucleic acid molecules in the sample (e.g., via complexation with a protein). As used herein, the term “does not substantially disrupt direct or indirect interactions between nucleic acid molecules or between regions of nucleic acid molecules” means that at least 5%, 10%, 15%, 20%, 25%, 30%, 40%, 50%, 60%, 70%, 80%, 90% or more of the physical associations between nucleic acid molecules of interest or between nucleic acid molecule regions of interest (e.g., nucleic acid molecules or nucleic acid regions to be detected according to the methods described herein) remain intact after extraction or isolation from the sample relative to the physical associations of such nucleic acid molecules or nucleic acid regions prior to extraction or isolation from the sample. In some embodiments, the extent to which extraction or isolation disrupts direct or indirect interactions for a sample can be measured and/or quantified by comparing a cross-linked control sample to a non-cross-linked sample. Chemical cross-linking methods are known in the art. See, e.g., Steen and Jensen, “Analysis of protein-nucleic acid interactions by photochemical cross-linking and mass spectrometry,” Mass Spectrom Rev. (2002) 21:163-82; Verdine and Normal, “Covalent trapping of protein-DNA complexes,” Annu Rev Biochem (2003) 72:337-66; and Chemistry of Protein and Nucleic Acid Cross-Linking and Conjugation, Second Edition, Wong and Jameson, Eds., CRC Press (2011).

In some embodiments, the sample can be prepared to facilitate or improve the detection of direct or indirect physical interactions. For example, in some embodiments the sample can be fragmented, fractionated, homogenized, or sonicated. Samples can be fragmented, fractionated, homogenized, or sonicated as desired. Exemplary methods are described in Ausubel et al., Current Protocols in Molecular Biology (1994); Sambrook and Russell, “Fragmentation of DNA by sonication,” Cold Spring Harbor Protocols (2006); and Burden, “Guide to the Homogenization of Biological Samples,” Random Primers (2008), pages 1-14.

In some embodiments, the sample comprises nucleic acid molecules or regions of a nucleic acid molecule in a complex with one or more other components, e.g., a protein, and the step of providing a mixture of nucleic acids comprises providing the mixture of nucleic acids under conditions such that proteins remain bound to the nucleic acid molecules or regions of the nucleic acid molecule in the mixture. In some embodiments, the nucleic acids are extracted or isolated in the presence of a salt (e.g., NaCl or KCl) at a concentration that supports the binding of proteins to nucleic acids in a complex. In some embodiments, the nucleic acids are extracted or isolated in the absence of an agent that denatures protein (e.g., in the absence of phenol, guanidine thiocyanate, or an anionic detergent).

In some embodiments, nucleic acid molecules or regions of nucleic acid molecules, or sub-fractions comprising target nucleic acid molecules or regions of nucleic acid molecules, are extracted or isolated from a sample comprising one or more cells by disrupting or dissolving the cell membrane of the cells. The term “disrupting” a cell membrane, as used herein, refers to reducing the integrity of a cell membrane such that the cell's structure does not remain intact. For example, contacting a cell membrane with a nonionic detergent will remove and/or dissolve a cell membrane. Cell membranes can be disrupted or dissolved as desired. As a non-limiting example, cell membranes can be disrupted using one or more non-ionic detergents. Exemplary non-ionic detergents include, but are not limited to, NP40, Tween20, and Triton X-100.

In some embodiments, a sample comprising one or more cells is permeabilized prior to extraction or isolation of the nucleic acids. As used herein, the term “permeabilizing” refers to reducing the integrity of a cell membrane to allow for entry of a nucleic acid cleaving or modifying agent (e.g., an enzyme) into the cell. A cell with a permeabilized cell membrane will generally retain the cell membrane such that the cell's structure remains substantially intact. For example, a cell can be permeabilized prior to treating or manipulating nucleic acids inside the cell (e.g., with an enzyme). Cell membranes can be permeabilized as desired. As a non-limiting example, cell membranes can be permeabilized using one or more lysolipids. Exemplary lysolipids include, but are not limited to, lysophosphatidylcholine (also known in the art as lysolecithin) or monopalmitoylphosphatidylcholine. A variety of lysolipids are also described in, e.g., WO 2003/052095. Alternatively, electroporation or biolistic methods can be used to permeabilize a cell membrane. A wide variety of electroporation methods are well known in the art, including, but are not limited to, those described in WO 2000/062855. Biolistic methods include but are not limited to those described in U.S. Pat. No. 5,179,022.

In some embodiments, the providing of nucleic acids further comprises digesting, cutting, or shearing the nucleic acids. In some embodiments, a sample (e.g., a sample comprising one or more cells) is permeabilized prior to digesting, cutting, or shearing the nucleic acids. Nucleic acid digestion, cutting, or shearing can be performed as desired. As a non-limiting example, an enzyme that digests or cuts nucleic acid molecules can be used. In some embodiments, the enzyme is an endoribonuclease, or “RNase.” Examples of suitable RNases include, but are not limited to, RNase H (i.e., RNase H, RNase H1, and RNase H2) and RNase A. RNases used can include naturally occurring RNases, recombinant RNases, and modified RNases (e.g., RNases comprising mutations, insertions, or deletions). In some embodiments, the enzyme is a ribozyme, an enzymatic RNA molecule capable of catalyzing the specific cleavage of RNA. Suitable ribozymes include both naturally occurring ribozymes and synthetic ribozymes. See, e.g., Heidenreich et al., Nucleic Acids Res., 23:2223-2228 (1995). In some embodiments, the enzyme an enzyme that cuts or digests DNA, or “DNase.” Examples of suitable DNases include, but are not limited to, micrococcal nuclease, S1 nuclease, P1 nuclease, mung bean nuclease, DNase I, and Bal 31 nuclease. As another non-limiting example, nucleic acids (e.g., DNA or RNA) can be sheared using a sonicator (e.g., Bioruptor® sonication device, Diagenode, Denville, N.J.). In some embodiments, the sample is treated with an enzyme (e.g., nuclease) that cuts or digests nucleic acid molecules in a sequence non-specific manner. In some embodiments, the sample is not treated with a sequence-specific restriction enzyme. In some embodiments, the sample is not treated with a methylation sensitive enzyme and/or is not treated with a methylating agent (e.g., a DNA methyltransferase).

In some embodiments, nucleic acids from the sample are extracted or isolated without a prior step of manipulating or treating the nucleic acids (e.g., digesting, cutting, or shearing the nucleic acids). In some embodiments, nucleic acids that have been extracted or isolated from the sample are subsequently manipulated or treated, e.g., by digesting, cutting, or shearing the nucleic acids, to facilitate detection of the nucleic acids.

In some embodiments, the nucleic acids are purified from other components in the sample. Purification procedures can be used to isolate a desired portion of the sample comprising the nucleic acids or to remove an unwanted portion from the sample. As a non-limiting example, a sample comprising an increased proportion of a desired protein (e.g., a protein that forms a complex with nucleic acids of interest), nucleic acid, or nucleic acid-protein complex can be isolated from a crude cell. In some aspects, for example, immunoprecipitation with an appropriate antibody can be performed to increase the proportion of the desired protein. Nucleic acid sequences can be enriched, for example, using a complementary nucleic acid sequence that forms a complex with the target sequence, with other sequences being separated from the target enriched sequence.

Essentially any nucleic acid purification procedure can be used so long as it results in nucleic acid molecules of acceptable purity for the subsequent detecting step. For example, standard cell lysis reagents can be used to lyse cells. Optionally a protease (including but not limited to proteinase K) can be used. Nucleic acids can be isolated from the sample as desired. In some embodiments, phenol/chloroform extractions are used and the nucleic acids can be subsequently precipitated (e.g., by ethanol) and purified. Alternatively, nucleic acids can be isolated on a nucleic-acid binding column.

In some embodiments, the extracted or isolated nucleic acids are resuspended in a solution prior to the compartmentalizing step. In some embodiments, the mixture or solution to be compartmentalized further comprises one or more reagents for detecting the nucleic acid molecules or the regions of the nucleic acid molecule (e.g., oligonucleotide probes, labeled oligonucleotide probes, or other detectable agents as described herein), one or more buffers (e.g., aqueous buffers) and/or one or more additives (e.g., blocking agents or biopreservatives).

### Compartmentalization

The mixture comprising the nucleic acids to be detected is compartmentalized into a plurality of compartments. Compartments can include any of a number of types of compartments, including solid compartments (e.g., wells, tubes, microchannels, etc.) and fluid compartments (e.g., aqueous droplets within an oil phase). In some embodiments, the compartments are droplets. In some embodiments, the compartments are microchannels. Methods and compositions for compartmentalizing a sample are described, for example, in published patent applications WO 2010/036352, US 2010/0173394, US 2011/0092373, and US 2011/0092376, the entire content of each of which is incorporated by reference herein.

In some embodiments, the compartments have an average volume of about 0.001 nL, about 0.005 nL, about 0.01 nL, about 0.02 nL, about 0.03 nL, about 0.04 nL, about 0.05 nL, about 0.06 nL, about 0.07 nL, about 0.08 nL, about 0.09 nL, about 0.1 nL, about 0.2 nL, about 0.3 nL, about 0.4 nL, about 0.5 nL, about 0.6 nL, about 0.7 nL, about 0.8 nL, about 0.9 nL, about 1 nL, about 1.5 nL, about 2 nL, about 2.5 nL, about 3 nL, about 3.5 nL, about 4 nL, about 4.5 nL, about 5 nL, about 5.5 nL, about 6 nL, about 6.5 nL, about 7 nL, about 7.5 nL, about 8 nL, about 8.5 nL, about 9 nL, about 9.5 nL, about 10 nL, about 11 nL, about 12 nL, about 13 nL, about 14 nL, about 15 nL, about 16 nL, about 17 nL, about 18 nL, about 19 nL, about 20 nL, about 25 nL, about 30 nL, about 35 nL, about 40 nL, about 45 nL, about 50 nL, about 60 nL, about 70 nL, about 80 nL, about 90 nL, 0.1 μl, about 0.5 μl, about 1 μl, about 2 μl, about 3 μl, about 4 μl, about 5 μl, about 6 μl, about 7 μl, about 8 μl, about 9 μl, about 10 μl, about 15 μl, about 20 μl, about 25 μl, about 30 μl, about 40 μl, about 50 μl, about 60 μl, about 70 μl, about 80 μl, about 90 μl, about 100 μl, about 150 μl, about 200 μl, about 250 μl, about 300 μl, about 350 μl, about 400 μl, about 450 μl, or about 500 μl. In some embodiments, the compartments have an average volume from about 0.1 nl to about 10 nl, from about 0.5 nl to about 5 nl, from about 1 nl to about 10 nl, from about 1 nl to about 50 nl, from about 5 nl to about 50 nl, from about 10 nl to about 50 nl, from about 10 nl to about 100 nl, from about 50 nl to about 500 nl, from about 0.1 μl to about 5 μl, from about 0.5 μl to about 5 μl, from about 0.5 μl to about 10 μl, from about 1 μl to about 5 μl, from about 1 μl to about 50 μl, from about 10 μl to about 50 μl, from about 10 μl to about 100 μl, from about 50 μl to about 100 μl, from about 50 μl to about 250 μl, from about 100 μl to about 250 μl, from about 100 μl to about 500 μl, or from about 250 μl to about 500 μl.

In some embodiments, the mixture comprising the nucleic acids is compartmentalized into a sufficient number of compartments such that co-localization of the nucleic acids due to close proximity can be distinguished from random co-localization. In some embodiments, the mixture comprising the nucleic acids is compartmentalized into at least 500 compartments, at least 1000 compartments, at least 2000 compartments, at least 3000 compartments, at least 4000 compartments, at least 5000 compartments, at least 6000 compartments, at least 7000 compartments, at least 8000 compartments, at least 10,000 compartments, at least 15,000 compartments, at least 20,000 compartments, at least 30,000 compartments, at least 40,000 compartments, at least 50,000 compartments, at least 60,000 compartments, at least 70,000 compartments, at least 80,000 compartments, at least 90,000 compartments, at least 100,000 compartments, at least 200,000 compartments, at least 300,000 compartments, at least 400,000 compartments, at least 500,000 compartments, at least 600,000 compartments, at least 700,000 compartments, at least 800,000 compartments, at least 900,000 compartments, at least 1,000,000 compartments, at least 2,000,000 compartments, at least 3,000,000 compartments, at least 4,000,000 compartments, at least 5,000,000 compartments, at least 10,000,000 compartments, at least 20,000,000 compartments, at least 30,000,000 compartments, at least 40,000,000 compartments, at least 50,000,000 compartments, at least 60,000,000 compartments, at least 70,000,000 compartments, at least 80,000,000 compartments, at least 90,000,000 compartments, at least 100,000,000 compartments, at least 150,000,000 compartments, or at least 200,000,000 compartments.

In some embodiments, the mixture comprising the nucleic acids is compartmentalized by aliquoting the mixture into a plurality of compartments. In some embodiments, the mixture is aliquoted into compartments on multi-well plates, e.g., on 48-, 96-, or 384-well plates. As a non-limiting example, the mixture can be aliquoted using an automated system such as the Freedom EVO® liquid handling system (Tecan Systems, Inc., San Jose, Calif.).

In some embodiments, the mixture comprising the nucleic acids is compartmentalized by dilution. Dilution can be achieved by physically diluting a sample to different extents, or by virtual dilution by changing the volume assayed in each compartment. In some embodiments, compartments of two or more sizes are generated. For example, a device that compartmentalizes the mixture into two or more compartment sizes, such as a droplet generator that produces at least two different sizes of monodisperse droplets, an emulsion that generates polydisperse droplets, or a plate with at least two volumes for compartmentalizing the sample, can be used.

In some embodiments, the number of compartments that is sufficient to distinguish co-localization of nucleic acids due to close proximity from random co-localization can be determined by serial dilution. For example, in some embodiments, the mixture is subdivided with some subdivisions being subsequently diluted further, thereby providing a mechanism to distinguish specific from random co-localization. If a particular subdivision is diluted into a larger number of subdivisions, the number of co-localizations due to nucleic acids in close proximity should stay the same but the number of random co-localizations should decrease by an amount predictable by the dilution factor and number of compartments. Although the frequency of co-localization due to nucleic acids in close proximity should decrease as well, the co-localization due to nucleic acids in close proximity only decreases in frequency in a manner predictable by the dilution factor and does not decrease in absolute amount, but the random co-localization will decrease by a much higher factor and thus serves as a mechanism to distinguish nucleic acid interactions from random co-localization.

In some embodiments, the mixture comprising the nucleic acids is compartmentalized using limiting dilution. Methods for quantitating nucleic acid targets using limiting dilution and PCR analysis are described, for example, in Sykes et al., Biotechniques 13:444-449 (1992). Briefly, in limiting dilution a series of sequential dilutions is performed on a sample (e.g., a mixture comprising nucleic acids) to create a dilution series. For example, a mixture comprising nucleic acids can be diluted in a solution (e.g., an aqueous buffer) to form a first dilution, which is then diluted to form a second dilution, which is then diluted to form a third, dilution, etc. Each dilution in the dilution series is compartmentalized into a plurality of compartments as described herein. The compartments are then assayed to identify a dilution at which co-localization of two or more non-interacting molecules in the compartment is unlikely to occur by random chance. Thus, the detection of co-localization of nucleic acids at such a dilution would be indicative of close proximity (e.g., direct or indirect physical interaction) between the nucleic acids.

Droplets

In some embodiments, the mixture is compartmentalized by droplet formation into a plurality of droplets. In some embodiments, a droplet comprises an emulsion composition, i.e., a mixture of immiscible fluids (e.g., water and oil). In some embodiments, a droplet is an aqueous droplet that is surrounded by an immiscible carrier fluid (e.g., oil). In some embodiments, a droplet is an oil droplet that is surrounded by an immiscible carrier fluid (e.g., an aqueous solution). In some embodiments, the droplets described herein are relatively stable and have minimal coalescence between two or more droplets. In some embodiments, less than 0.0001%, 0.0005%, 0.001%, 0.005%, 0.01%, 0.05%, 0.1%, 0.5%, 1%, 2%, 3%, 4%, 5%, 6%, 7%, 8%, 9%, or 10% of droplets generated from a sample coalesce with other droplets. The emulsions can also have limited flocculation, a process by which the dispersed phase comes out of suspension in flakes.

In some embodiments, the droplets that are generated are substantially uniform in volume. For example, in some embodiments, the droplets that are generated have an average volume of about 0.001 nL, about 0.005 nL, about 0.01 nL, about 0.02 nL, about 0.03 nL, about 0.04 nL, about 0.05 nL, about 0.06 nL, about 0.07 nL, about 0.08 nL, about 0.09 nL, about 0.1 nL, about 0.2 nL, about 0.3 nL, about 0.4 nL, about 0.5 nL, about 0.6 nL, about 0.7 nL, about 0.8 nL, about 0.9 nL, about 1 nL, about 1.5 nL, about 2 nL, about 2.5 nL, about 3 nL, about 3.5 nL, about 4 nL, about 4.5 nL, about 5 nL, about 5.5 nL, about 6 nL, about 6.5 nL, about 7 nL, about 7.5 nL, about 8 nL, about 8.5 nL, about 9 nL, about 9.5 nL, about 10 nL, about 11 nL, about 12 nL, about 13 nL, about 14 nL, about 15 nL, about 16 nL, about 17 nL, about 18 nL, about 19 nL, about 20 nL, about 25 nL, about 30 nL, about 35 nL, about 40 nL, about 45 nL, about 50 nL, about 60 nL, about 70 nL, about 80 nL, about 90 nL, about 100 nL, about 0.2 μL, about 0.3 μL, about 0.4 μL, about 0.5 μL, about 0.6 μL, about 0.7 μL, about 0.8 μL, about 0.9 μL, about 1 μL, about 1.5 μL, about 2 μL, about 2.5 μL, about 3 μL, about 3.5 μL, about 4 μL, about 4.5 μL, about 5 μL, about 5.5 μL, about 6 μL, about 6.5 μL, about 7 μL, about 7.5 μL, about 8 μL, about 8.5 μL, about 9 μL, about 9.5 μL, about 10 μL, about 11 μL, about 12 μL, about 13 μL, about 14 μL, about 15 μL, about 16 μL, about 17 μL, about 18 μL, about 19 μL, about 20 μL, about 25 μL, about 30 μL, about 35 μL, about 40 μL, about μL, about 50 μL, about 60 μL, about 70 μL, about 80 μL, about 90 μL, about 100 μL, about 150 μL, about 200 μL, about 250 μL, about 300 μL, about 350 μL, about 400 μL, about 450 μL, or about 500 μL.

In some embodiments, the droplet is formed by flowing an oil phase through an aqueous sample comprising the nucleic acids to be detected. In some embodiments, the aqueous sample comprising the nucleic acids to be detected further comprises a buffered solution and one or more reagents (e.g., reagents for amplification of the nucleic acids, such as oligonucleotide probes or labeled oligonucleotide probes, or other detectable agents as described herein) for detecting the nucleic acids.

The oil phase may comprise a fluorinated base oil which may additionally be stabilized by combination with a fluorinated surfactant such as a perfluorinated polyether. In some embodiments, the base oil comprises one or more of a HFE 7500, FC-40, FC-43, FC-70, or another common fluorinated oil. In some embodiments, the oil phase comprises an anionic fluorosurfactant. In some embodiments, the anionic fluorosurfactant is Ammonium Krytox (Krytox-AS), the ammonium salt of Krytox FSH, or a morpholino derivative of Krytox FSH. Krytox-AS may be present at a concentration of about 0.1%, 0.2%, 0.3%, 0.4%, 0.5%, 0.6%, 0.7%, 0.8%, 0.9%, 1.0%, 2.0%, 3.0%, or 4.0% (w/w). In some embodiments, the concentration of Krytox-AS is about 1.8%. In some embodiments, the concentration of Krytox-AS is about 1.62%. Morpholino derivative of Krytox FSH may be present at a concentration of about 0.1%, 0.2%, 0.3%, 0.4%, 0.5%, 0.6%, 0.7%, 0.8%, 0.9%, 1.0%, 2.0%, 3.0%, or 4.0% (w/w). In some embodiments, the concentration of morpholino derivative of Krytox FSH is about 1.8%. In some embodiments, the concentration of morpholino derivative of Krytox FSH is about 1.62%.

In some embodiments, the oil phase further comprises an additive for tuning the oil properties, such as vapor pressure, viscosity, or surface tension. Non-limiting examples include perfluorooctanol and 1H,1H,2H,2H-Perfluorodecanol. In some embodiments, 1H,1H,2H,2H-Perfluorodecanol is added to a concentration of about 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.25%, 1.50%, 1.75%, 2.0%, 2.25%, 2.5%, 2.75%, or 3.0% (w/w). In some embodiments, 1H,1H,2H,2H-Perfluorodecanol is added to a concentration of about 0.18% (w/w).

In some embodiments, the emulsion is formulated to produce highly monodisperse droplets having a liquid-like interfacial film that can be converted by heating into microcapsules having a solid-like interfacial film; such microcapsules may behave as bioreactors able to retain their contents through an incubation period. The conversion to microcapsule form may occur upon heating. For example, such conversion may occur at a temperature of greater than about 40°, 50°, 60°, 70°, 80°, 90°, or 95° C. During the heating process, a fluid or mineral oil overlay may be used to prevent evaporation. Excess continuous phase oil may or may not be removed prior to heating. The biocompatible capsules may be resistant to coalescence and/or flocculation across a wide range of thermal and mechanical processing.

Following conversion, the microcapsules may be stored at about −70°, −20°, 0°, 3°, 4°, 5°, 6°, 7°, 8°, 9°, 10°, 15°, 20°, 25°, 30°, 35°, or 40° C. In some embodiments, these capsules are useful in biomedical applications, such as stable, digitized encapsulation of macromolecules, particularly aqueous biological fluids comprising a mix of target molecules such as nucleic acids, proteins, or both together; drug and vaccine delivery; biomolecular libraries; clinical imaging applications; and others.

The microcapsule compartments may resist coalescence, particularly at high temperatures. Accordingly, the capsules can be incubated at a very high density (e.g., number of compartments per unit volume). In some embodiments, greater than 100,000, 500,000, 1,000,000, 1,500,000, 2,000,000, 2,500,000, 5,000,000, or 10,000,000 compartments may be incubated per mL. In some embodiments, the microcapsules also contain other components such as reagents for amplification of the nucleic acids (e.g., oligonucleotide probes or labeled oligonucleotide probes).

### Detection

A variety of methods can be used to detect and/or quantify the extent to which nucleic acids in a sample are in close proximity to each other. In some embodiments, detecting the presence of two or more nucleic acid molecules or two or more regions of a nucleic acid molecule in the same compartment comprises amplifying the nucleic acid molecules or regions of the nucleic acid molecule. In some embodiments, detecting the presence of two or more nucleic acid molecules or two or more regions of a nucleic acid molecule in the same compartment comprises nucleotide sequencing the nucleic acid molecules or regions of the nucleic acid molecule. In some embodiments, detecting the presence of two or more nucleic acid molecules or two or more regions of a nucleic acid molecule in the same compartment comprises detecting one or more agents that hybridize to the nucleic acid molecules or to the regions of the nucleic acid molecule, or that specifically associate with the nucleic acid molecules or regions of the nucleic acid molecule (e.g., by specifically binding to a component of a complex comprising the nucleic acids, such as a protein-nucleic acid complex).

Amplification

In some embodiments, the detecting step comprises amplifying the nucleic acid molecules or regions of the nucleic acid molecule. In some embodiments, amplifying the nucleic acid molecules or regions of the nucleic acid molecule comprises polymerase chain reaction (PCR), quantitative PCR, or real-time PCR.

As discussed below, quantitative amplification (including, but not limited to, real-time PCR) methods allow for determination of the amount of nucleic acid molecules or regions of a nucleic acid molecule that co-localize in a compartment, and can be used with various controls to determine the relative amount of co-localization of nucleic acid molecules or regions of a nucleic acid molecule in a sample of interest, thereby indicating whether and to what extent nucleic acids in a sample are in close proximity to each other.

Quantitative amplification methods (e.g., quantitative PCR or quantitative linear amplification) involve amplification of nucleic acid template, directly or indirectly (e.g., determining a Ct value) determining the amount of amplified DNA, and then calculating the amount of initial template based on the number of cycles of the amplification. Amplification of a DNA locus using reactions is well known (see U.S. Pat. Nos. 4,683,195 and 4,683,202; PCR PROTOCOLS: A GUIDE TO METHODS AND APPLICATIONS (Innis et al., eds, 1990)). Typically, PCR is used to amplify DNA templates. However, alternative methods of amplification have been described and can also be employed. Methods of quantitative amplification are disclosed in, e.g., U.S. Pat. Nos. 6,180,349; 6,033,854; and 5,972,602, as well as in, e.g., Gibson et al., Genome Research 6:995-1001 (1996); DeGraves, et al., Biotechniques 34(1):106-10, 112-5 (2003); Deiman B, et al., Mol Biotechnol. 20(2):163-79 (2002). Amplifications can be monitored in “real time.”

In some embodiments, quantitative amplification is based on the monitoring of the signal (e.g., fluorescence of a probe) representing copies of the template in cycles of an amplification (e.g., PCR) reaction. In the initial cycles of the PCR, a very low signal is observed because the quantity of the amplicon formed does not support a measurable signal output from the assay. After the initial cycles, as the amount of formed amplicon increases, the signal intensity increases to a measurable level and reaches a plateau in later cycles when the PCR enters into a non-logarithmic phase. Through a plot of the signal intensity versus the cycle number, the specific cycle at which a measurable signal is obtained from the PCR reaction can be deduced and used to back-calculate the quantity of the target before the start of the PCR. The number of the specific cycles that is determined by this method is typically referred to as the cycle threshold (Ct). Exemplary methods are described in, e.g., Heid et al. Genome Methods 6:986-94 (1996) with reference to hydrolysis probes.

One method for detection of amplification products is the 5′-3′ exonuclease “hydrolysis” PCR assay (also referred to as the TaqMan™ assay) (U.S. Pat. Nos. 5,210,015 and 5,487,972; Holland et al., PNAS USA 88: 7276-7280 (1991); Lee et al., Nucleic Acids Res. 21: 3761-3766 (1993)). This assay detects the accumulation of a specific PCR product by hybridization and cleavage of a doubly labeled fluorogenic probe (the TaqMan™ probe) during the amplification reaction. The fluorogenic probe consists of an oligonucleotide labeled with both a fluorescent reporter dye and a quencher dye. During PCR, this probe is cleaved by the 5′-exonuclease activity of DNA polymerase if, and only if, it hybridizes to the segment being amplified. Cleavage of the probe generates an increase in the fluorescence intensity of the reporter dye.

Another method of detecting amplification products that relies on the use of energy transfer is the “beacon probe” method described by Tyagi and Kramer, Nature Biotech. 14:303-309 (1996), which is also the subject of U.S. Pat. Nos. 5,119,801 and 5,312,728. This method employs oligonucleotide hybridization probes that can form hairpin structures. On one end of the hybridization probe (either the 5′ or 3′ end), there is a donor fluorophore, and on the other end, an acceptor moiety. In the case of the Tyagi and Kramer method, this acceptor moiety is a quencher, that is, the acceptor absorbs energy released by the donor, but then does not itself fluoresce. Thus, when the beacon is in the open conformation, the fluorescence of the donor fluorophore is detectable, whereas when the beacon is in hairpin (closed) conformation, the fluorescence of the donor fluorophore is quenched. When employed in PCR, the molecular beacon probe, which hybridizes to one of the strands of the PCR product, is in the open conformation and fluorescence is detected, while those that remain unhybridized will not fluoresce (Tyagi and Kramer, Nature Biotechnol. 14: 303-306 (1996)). As a result, the amount of fluorescence will increase as the amount of PCR product increases, and thus may be used as a measure of the progress of the PCR. Those of skill in the art will recognize that other methods of quantitative amplification are also available.

Various other techniques for performing quantitative amplification of nucleic acids are also known. For example, some methodologies employ one or more probe oligonucleotides that are structured such that a change in fluorescence is generated when the oligonucleotide(s) is hybridized to a target nucleic acid. For example, one such method involves is a dual fluorophore approach that exploits fluorescence resonance energy transfer (FRET), e.g., LightCycler™ hybridization probes, where two oligo probes anneal to the amplicon. The oligonucleotides are designed to hybridize in a head-to-tail orientation with the fluorophores separated at a distance that is compatible with efficient energy transfer. Other examples of labeled oligonucleotides that are structured to emit a signal when bound to a nucleic acid or incorporated into an extension product include: Scorpions™ probes (e.g., Whitcombe et al., Nature Biotechnology 17:804-807, 1999, and U.S. Pat. No. 6,326,145), Sunrise™ (or Amplifluor™) probes (e.g., Nazarenko et al., Nuc. Acids Res. 25:2516-2521, 1997, and U.S. Pat. No. 6,117,635), and probes that form a secondary structure that results in reduced signal without a quencher and that emits increased signal when hybridized to a target (e.g., Lux Probes™).

Nucleotide Sequencing

In some embodiments, the detecting step comprises nucleotide sequencing the nucleic acid molecules or regions of the nucleic acid molecule. Non-limiting examples of nucleotide sequencing include Sanger sequencing, capillary array sequencing, thermal cycle sequencing (Sears et al., Biotechniques 13:626-633 (1992)), solid-phase sequencing (Zimmerman et al., Methods Mol. Cell Biol. 3:39-42 (1992)), sequencing with mass spectrometry such as matrix-assisted laser desorption/ionization time-of-flight mass spectrometry (MALDI-TOF/MS; Fu et al., Nature Biotech. 16:381-384 (1998)), and sequencing by hybridization (Chee et al., Science 274:610-614 (1996); Drmanac et al., Science 260:1649-1652 (1993); Drmanac et al., Nature Biotech. 16:54-58 (1998)). In some embodiments, “next generation sequencing” methods can be used, for example but not limited to, sequencing by synthesis (e.g., HiSeq™, MiSeg™, or Genome Analyzer, each available from Illumina), sequencing by ligation (e.g., SOLiD™, Life Technologies), ion semiconductor sequencing (e.g., Ion Torrent™, Life Technologies), and pyrosequencing (e.g., 454™ sequencing, Roche Diagnostics). In some embodiments, nucleotide sequencing comprises high-throughput sequencing. In high-throughput sequencing, parallel sequencing reactions using multiple templates and multiple primers allows rapid sequencing of genomes or large portions of genomes. See, e.g., WO 03/004690, WO 03/054142, WO 2004/069849, WO 2004/070005, WO 2004/070007, WO 2005/003375, WO 2000/006770, WO 2000/027521, WO 2000/058507, WO 2001/023610, WO 2001/057248, WO 2001/057249, WO 2002/061127, WO 2003/016565, WO 2003/048387, WO 2004/018497, WO 2004/018493, WO 2004/050915, WO 2004/076692, WO 2005/021786, WO 2005/047301, WO 2005/065814, WO 2005/068656, WO 2005/068089, WO 2005/078130, and Seo, et al., Proc. Natl. Acad. Sci. USA (2004) 101:5488-5493.

In some embodiments, nucleotide sequencing comprises single-molecule, real-time (SMRT) sequencing. SMRT sequencing is a process by which single DNA polymerase molecules are observed in real time while they catalyze the incorporation of fluorescently labeled nucleotides complementary to a template nucleic acid strand. Methods of SMRT sequencing are known in the art and were initially described by Flusberg et al., Nature Methods, 7:461-465 (2010), which is incorporated herein by reference for all purposes. Briefly, in SMRT sequencing, incorporation of a nucleotide is detected as a pulse of fluorescence whose color identifies that nucleotide. The pulse ends when the fluorophore, which is linked to the nucleotide's terminal phosphate, is cleaved by the polymerase before the polymerase translocates to the next base in the DNA template. Fluorescence pulses are characterized by emission spectra as well as by the duration of the pulse (“pulse width”) and the interval between successive pulses (“interpulse duration” or “IPD”). Pulse width is a function of all kinetic steps after nucleotide binding and up to fluorophore release, and IPD is a function of the kinetics of nucleotide binding and polymerase translocation. Thus, DNA polymerase kinetics can be monitored by measuring the fluorescence pulses in SMRT sequencing.

In addition to measuring differences in fluorescence pulse characteristics for each fluorescently-labeled nucleotide (i.e., adenine, guanine, thymine, and cytosine), differences can also be measured for non-methylated versus methylated bases. For example, the presence of a methylated base alters the IPD of the methylated base as compared to its non-methylated counterpart (e.g., methylated adenosine as compared to non-methylated adenosine). Additionally, the presence of a methylated base alters the pulse width of the methylated base as compared to its non-methylated counterpart (e.g., methylated cytosine as compared to non-methylated cytosine) and furthermore, different modifications have different pulse widths (e.g., 5-hydroxymethylcytosine has a more pronounced excursion than 5-methylcytosine). Thus, each type of non-modified base and modified base has a unique signature based on its combination of IPD and pulse width in a given context. The sensitivity of SMRT sequencing can be further enhanced by optimizing solution conditions, polymerase mutations and algorithmic approaches that take advantage of the nucleotides' kinetic signatures, and deconvolution techniques to help resolve neighboring methylcytosine bases.

In some embodiments, nucleotide sequencing comprises nanopore sequencing. Nanopore sequencing is a process by which a polynucleotide or nucleic acid fragment is passed through a pore (such as a protein pore) under an applied potential while recording modulations of the ionic current passing through the pore. Methods of nanopore sequencing are known in the art; see, e.g., Clarke et al., Nature Nanotechnology 4:265-270 (2009), which is incorporated herein by reference for all purposes. Briefly, in nanopore sequencing, as a single-stranded DNA molecule passes through a protein pore, each base is registered, in sequence, by a characteristic decrease in current amplitude which results from the extent to which each base blocks the pore. An individual nucleobase can be identified on a static strand, and by sufficiently slowing the rate of speed of the DNA translocation (e.g., through the use of enzymes) or improving the rate of DNA capture by the pore (e.g., by mutating key residues within the protein pore), an individual nucleobase can also be identified while moving.

In some embodiments, nanopore sequencing comprises the use of an exonuclease to liberate individual nucleotides from a strand of DNA, wherein the bases are identified in order of release, and the use of an adaptor molecule that is covalently attached to the pore in order to permit continuous base detection as the DNA molecule moves through the pore. As the nucleotide passes through the pore, it is characterized by a signature residual current and a signature dwell time within the adapter, making it possible to discriminate between non-methylated nucleotides. Additionally, different dwell times are seen between methylated nucleotides and the corresponding non-methylated nucleotides (e.g., 5-methyl-dCMP has a longer dwell time than dCMP), thus making it possible to simultaneously determine nucleotide sequence and whether sequenced nucleotides are modified. The sensitivity of nanopore sequencing can be further enhanced by optimizing salt concentrations, adjusting the applied potential, pH, and temperature, or mutating the exonuclease to vary its rate of processivity.

Agents for Detecting Nucleic Acids

In some embodiments, the detecting step comprises detecting one or more agents that hybridize to the nucleic acid molecules or to the regions of the nucleic acid molecule, or that specifically binds to a component that is complexed with the nucleic acid molecules or regions of the nucleic acid molecule. In some embodiments, the agent is a detectable agent.

In some embodiments, the method comprises contacting the nucleic acids with 1, 2, 3, 4, 5 or more agents, wherein each agent hybridizes to a different nucleic acid molecule or region of the nucleic acid molecule, and detecting the presence of the 1, 2, 3, 4, 5 or more agents; thereby detecting an interaction between the nucleic acid molecules or between the regions of the nucleic acid molecule in the sample. In some embodiments, the method comprises contacting the nucleic acids with at least two agents, wherein the first agent hybridizes to a first nucleic acid molecule or a first region of a nucleic acid molecule and wherein the second agent hybridizes to a second nucleic acid molecule or a second region of a nucleic acid molecule; and detecting the presence of the first agent and the second agent; thereby detecting an interaction between the two or more nucleic acid molecules or between the two or more regions of the nucleic acid molecule in the sample. In some embodiments, the first agent and the second agent combine to produce a signal that is not generated in the absence of the first agent and/or the second agent.

In some embodiments, the nucleic acids are detected by detecting one or more agents that specifically bind to a protein that specifically associates with the nucleic acid molecules or regions of the nucleic acid molecule in a complex. In some embodiments, the agent is an antibody that specifically binds to the protein.

In some embodiments, the agent comprises an optically detectable agent such as a fluorescent agent, phosphorescent agent, chemiluminescent agent, etc. Numerous agents (e.g., dyes, probes, or indicators) are known in the art and can be used in the present invention. (See, e.g., Invitrogen, The Handbook—A Guide to Fluorescent Probes and Labeling Technologies, Tenth Edition (2005)). Fluorescent agents can include a variety of organic and/or inorganic small molecules or a variety of fluorescent proteins and derivatives thereof. In some embodiments, the agent is a fluorophore. A vast array of fluorophores are reported in the literature and thus known to those skilled in the art, and many are readily available from commercial suppliers to the biotechnology industry. Literature sources for fluorophores include Cardullo et al., Proc. Natl. Acad. Sci. USA 85: 8790-8794 (1988); Dexter, D. L., J. of Chemical Physics 21: 836-850 (1953); Hochstrasser et al., Biophysical Chemistry 45: 133-141 (1992); Selvin, P., Methods in Enzymology 246: 300-334 (1995); Steinberg, I. Ann. Rev. Biochem., 40: 83-114 (1971); Stryer, L. Ann. Rev. Biochem., 47: 819-846 (1978); Wang et al., Tetrahedron Letters 31: 6493-6496 (1990); Wang et al., Anal. Chem. 67: 1197-1203 (1995). Non-limiting examples of fluorophores include cyanines, fluoresceins (e.g., 5′-carboxyfluorescein (FAM), Oregon Green, and Alexa 488), rhodamines (e.g., N,N,N′,N′-tetramethyl-6-carboxyrhodamine (TAMRA), tetramethyl rhodamine, and tetramethyl rhodamine isothiocyanate (TRITC)), eosin, coumarins, pyrenes, tetrapyrroles, arylmethines, oxazines, polymer dots, and quantum dots.

In some embodiments, the agent is an intercalating agent. Intercalating agents produce a signal when intercalated in double stranded nucleic acids. Exemplary agents include SYBR GREEN™, SYBR GOLD™, and EVAGREEN™.

In some embodiments, the agent is a molecular beacon oligonucleotide probe. As described above, the “beacon probe” method relies on the use of energy transfer. This method employs oligonucleotide hybridization probes that can form hairpin structures. On one end of the hybridization probe (either the 5′ or 3′ end), there is a donor fluorophore, and on the other end, an acceptor moiety. In the case of the Tyagi and Kramer method, this acceptor moiety is a quencher, that is, the acceptor absorbs energy released by the donor, but then does not itself fluoresce. Thus, when the beacon is in the open conformation, the fluorescence of the donor fluorophore is detectable, whereas when the beacon is in hairpin (closed) conformation, the fluorescence of the donor fluorophore is quenched.

In some embodiments, the agent is a radioisotope. Radioisotopes include radionuclides that emit gamma rays, positrons, beta and alpha particles, and X-rays. Suitable radionuclides include but are not limited to ²²⁵Ac, ⁷²As, ²¹¹At, ¹¹B, ¹²⁸Ba, ²¹²Bi, ⁷⁵Br, ⁷⁷Br, ¹⁴C, ¹⁰⁹Cd, ⁶²Cu, ⁶⁴Cu, ⁶⁷Cu, ¹⁸F, ⁶⁷Ga, ⁶⁸Ga, ³H, ¹⁶⁶Ho, ¹²³I, ¹²⁴I, ¹²⁵I, ¹³⁰I, ¹³¹I, ¹¹¹In, ¹⁷⁷Lu, ¹³N, ¹⁵O, ³²P, ³³P, ²¹²Pb, ¹⁰³Pd, ¹⁸⁶Re, ¹⁸⁸Re, ⁴⁷Sc, ¹⁵³Sm, ⁸⁹Sr, ^{99m}Tc, ⁸⁸Y and ⁹⁰Y.

In some embodiments, the agent is an enzyme, and the hybridization or specific association of the agent with the nucleic acid is detected by detecting a product generated by the enzyme. Examples of suitable enzymes include, but are not limited to, urease, alkaline phosphatase, (horseradish) hydrogen peroxidase (HRP), glucose oxidase, β-galactosidase, luciferase, alkaline phosphatase, and an esterase that hydrolyzes fluorescein diacetate. For example, a horseradish-peroxidase detection system can be used with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, which yields a soluble product readily detectable at 405 nm. A β-galactosidase detection system can be used with the chromogenic substrate o-nitrophenyl-β-D-galactopyranoside (ONPG), which yields a soluble product detectable at 410 nm. A urease detection system can be used with a substrate such as urea-bromocresol purple (Sigma Immunochemicals; St. Louis, Mo.).

In some embodiments, the agent is an oligonucleotide that is labeled with a detectable agent (e.g., an optical agent or radioisotope as described herein). The oligonucleotide hybridizes to the nucleic acid molecule or region of nucleic acid molecule of interest. In some embodiments, In some embodiments, the oligonucleotide is at least 5, 10, 15, 20, 25, 30, 35, 40, 45, 50, 60, 70, 80, 90, 100, 200, 300, 400, 500, 600, 700, 800, 900, 1000, 1500, 2000, 2500, 3000, 3500, 4000, 4500, 5000, or more nucleotides in length.

A detectable agent can be detected using any of a variety of detector devices. Exemplary detection methods include radioactive detection, optical absorbance detection (e.g., fluorescence or chemiluminescence), or mass spectral detection. As a non-limiting example, a fluorescent agent can be detected using a detector device equipped with a module to generate excitation light that can be absorbed by a fluorescer, as well as a module to detect light emitted by the fluorescer.

In some embodiments, the detectable agent in compartmentalized samples can be detected in bulk. For example, compartmentalized samples (e.g., droplets) can be compartmentalized into one or more wells of a plate, such as a 96-well or 384-well plate, and the signal(s) (e.g., fluorescent signal(s)) may be detected using a plate reader.

In some embodiments, the detector further comprises handling capabilities for the compartmentalized samples (e.g., droplets), with individual compartmentalized samples entering the detector, undergoing detection, and then exiting the detector. In some embodiments, compartmentalized samples (e.g., droplets) may be detected serially while the compartmentalized samples are flowing. In some embodiments, compartmentalized samples (e.g., droplets) are arrayed on a surface and a detector moves relative to the surface, detecting signal(s) at each position containing a single compartment. Examples of detectors are provided in WO 2010/036352, the contents of which are incorporated herein by reference. In some embodiments, detectable agents in compartmentalized samples can be detected serially without flowing the compartmentalized samples (e.g., using a chamber slide).

Following acquisition of fluorescence detection data, a general purpose computer system (referred to herein as a “host computer”) can be used to store and process the data. A computer-executable logic can be employed to perform such functions as subtraction of background signal, assignment of target and/or reference sequences, and quantification of the data. A host computer can be useful for displaying, storing, retrieving, or calculating diagnostic results from the molecular profiling; storing, retrieving, or calculating raw data from expression analysis; or displaying, storing, retrieving, or calculating any sample or patient information useful in the methods of the present invention.

The host computer may be configured with many different hardware components and can be made in many dimensions and styles (e.g., desktop PC, laptop, tablet PC, handheld computer, server, workstation, mainframe). Standard components, such as monitors, keyboards, disk drives, CD and/or DVD drives, and the like, may be included. Where the host computer is attached to a network, the connections may be provided via any suitable transport media (e.g., wired, optical, and/or wireless media) and any suitable communication protocol (e.g., TCP/IP); the host computer may include suitable networking hardware (e.g., modem, Ethernet card, WiFi card). The host computer may implement any of a variety of operating systems, including UNIX, Linux, Microsoft Windows, MacOS, or any other operating system.

Computer code for implementing aspects of the present invention may be written in a variety of languages, including PERL, C, C++, Java, JavaScript, VBScript, AWK, or any other scripting or programming language that can be executed on the host computer or that can be compiled to execute on the host computer. Code may also be written or distributed in low level languages such as assembler languages or machine languages.

The host computer system advantageously provides an interface via which the user controls operation of the tools. In the examples described herein, software tools are implemented as scripts (e.g., using PERL), execution of which can be initiated by a user from a standard command line interface of an operating system such as Linux or UNIX. Those skilled in the art will appreciate that commands can be adapted to the operating system as appropriate. In other embodiments, a graphical user interface may be provided, allowing the user to control operations using a pointing device. Thus, the present invention is not limited to any particular user interface.

Scripts or programs incorporating various features of the present invention may be encoded on various computer readable media for storage and/or transmission. Examples of suitable media include magnetic disk or tape, optical storage media such as compact disk (CD) or DVD (digital versatile disk), flash memory, and carrier signals adapted for transmission via wired, optical, and/or wireless networks conforming to a variety of protocols, including the Internet.

### Digital Analysis

In some embodiments, a digital readout assay, e.g., digital analysis, can be used to quantify the extent to which nucleic acids in a sample are in close proximity by compartmentalizing the mixture comprising the nucleic acids and identifying the compartments containing co-localized nucleic acids. Generally, the process of digital analysis involves determining for each compartment of a sample whether the compartment is positive or negative for the presence of the nucleic acid molecules or regions of the nucleic acid molecule to be detected. A compartment is “positive” if each of the nucleic acid molecules or regions of the nucleic acid molecule is detected in the compartment. In some embodiments, each of the nucleic acid molecules or regions of the nucleic acid molecule is detected in the compartment by detecting the presence of amplification products from both of the nucleic acid molecules or regions of the nucleic acid molecule (e.g., by detecting fluorescent signals associated with amplification reactions or products), or by detecting the presence of agents that hybridize to the nucleic acid molecules or regions of the nucleic acid molecule or associate in a complex with the nucleic acid molecules or regions of the nucleic acid molecule. A compartment is “negative” if at least one of the nucleic acid molecules or regions of the nucleic acid molecule is not detected in the compartment.

In some embodiments, a detector that is capable of detecting a signal or multiple signals is used to analyze each compartment for the presence or absence of the nucleic acid molecules or regions of the nucleic acid molecule. For example, in some embodiments a two-color reader (fluorescence detector) is used. The fraction of positive-counted compartments can enable the determination of an absolute amount of co-localization of nucleic acid molecules or regions of the nucleic acid molecule.

Once a binary “yes-no” result has been determined for each of the compartments of the sample, the data for the compartments is analyzed using an algorithm based on Poisson statistics to quantitate the amount of co-localization of nucleic acid molecules or regions of the nucleic acid molecule in the sample. Statistical methods for quantitating the concentration or amount of nucleic acids is described, for example, in WO 2010/036352, which is incorporated by reference herein in its entirety.

In some embodiments, a sample of interest that has been analyzed in each compartment for the presence or absence of the two or more nucleic acid molecules or two or more regions of the nucleic acid molecule is compared to a control to determine whether the number of positive compartments from the sample of interest is higher than the number of positive compartments from the control sample. In some embodiments, the control sample is a sample that has been treated to remove proteins from the sample or disrupt protein-nucleic acid interactions in the sample, e.g., through the use of buffers, enzymes, or heat inactivation. For example, in some embodiments, the control sample is a sample in which the nucleic acids have been extracted or isolated in a high salt buffer to disrupt nucleic acid-protein interactions. In some embodiments, the two or more nucleic acid molecules or the two or more regions of the nucleic acid molecule in the sample are determined to be in close proximity to each other due to indirect interactions (e.g., via complexation with a protein) when the number of positive compartments for the sample is at least two-fold, three-fold, four-fold, five-fold, six-fold, seven-fold, eight-fold, nine-fold, ten-fold or higher relative to the number of positive compartments obtained for a control sample that has been treated to remove proteins or disrupt protein-nucleic acid interactions in the sample.

### III. Kits

In another aspect, kits for determining whether two or more nucleic acid molecules or two or more regions of a nucleic acid molecule in a sample are in close proximity to each other are provided. Kits of the present invention can include, for example, reagents for detecting nucleic acid proximity as described herein (e.g., one or more reagents for sequencing the nucleic acids, one or more reagents for quantitatively amplifying the nucleic acids, or one or more detectable agents that hybridize to the nucleic acids or that specifically bind to a component that is complexed with the nucleic acids, e.g., oligonucleotide probes, labeled oligonucleotide probes, or other detectable agents as described herein). The kits can optionally include written instructions or electronic instructions (e.g., on a CD-ROM or DVD). In some embodiments, the kits further comprise an agent for disrupting, dissolving, or permeabilizing a cell membrane (e.g., a lysolipid or a non-ionic detergent). In some embodiments, the kits further comprise an agent for digesting, cutting, or shearing the nucleic acids (e.g, an enzyme such as an RNase or a DNase). In some embodiments, the kits further comprise reagents and/or materials for the extraction and/or purification of nucleic acids (e.g., cell lysis reagents or a nucleic acid binding column). In some embodiments, the kits further comprise reagents and/or materials for the compartmentalization of the mixtures comprising the nucleic acids.

The kits can also include one or more control samples. Exemplary control samples include, e.g., samples that are known to be positive for direct or indirect nucleic acid physical interactions, or samples that are known to be negative for direct or indirect nucleic acid physical interactions.

### IV. EXAMPLES

The following examples are offered to illustrate, but not to limit the claimed invention.

### Example 1

### Detecting Interactions Between Nucleic Acid Regions

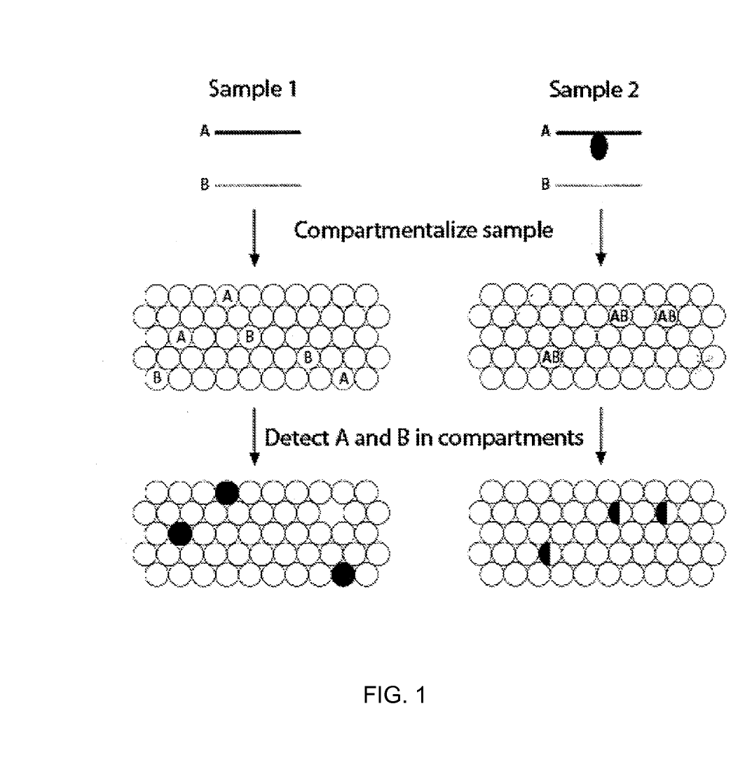

This example provides a method for determining if two nucleic acid regions (for example, DNA) directly or indirectly physically interact with each other. A schematic depicting this example is provided in FIG. 1. In Sample 1, DNA regions A and B are not proximal to each other and there is no interaction between them. In Sample 2, DNA regions A and B interact indirectly through proteins that are associated with them; thus, in Sample 2 DNA regions A and B are components of a larger protein:DNA complex that will segregate as a group.

If the samples were to be compartmentalized such that (a) the number of compartments is much greater than the number of A and B DNA molecules and (b) the physical size of the individual compartments is much bigger than the protein:DNA complex that contains the A and B DNA molecules, then in Sample 1, in most cases DNA regions A and B will partition into different compartments. In contrast, because in Sample 2 molecules A and B are part of the same protein:DNA complex, in most cases DNA regions A and B will partition into the same compartment.

If the individual compartments were then to be interrogated to determine if they contain DNA region A and/or B, then results from Sample 1 would show that DNA regions A and B would most often be found in separate compartments, but for Sample 2 DNA regions A and B would most often be found in the same compartment. From this data, one can infer that in Sample 1 DNA regions A and B are not physically associated with each other, whereas in Sample 2 DNA regions A and B are in close association. These results may provide valuable information regarding complex nucleic acid structures and interactions.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of determining whether two or more nucleic acid molecules or two or more regions of a nucleic acid molecule in a sample are in close proximity to each other, the method comprising:
providing a mixture of nucleic acids;
compartmentalizing the mixture into a sufficient number of compartments such that co-localization in a compartment of nucleic acid molecules due to close proximity can be distinguished from random co-localization; and
detecting the presence of two or more nucleic acid molecules or two or more regions of a nucleic acid molecule in the same compartment; thereby determining that the two or more nucleic acid molecules or the two or more regions of the nucleic acid molecule in the sample are in close proximity to each other.

2. The method of claim 1, wherein two or more nucleic acid molecules are detected.

3. The method of claim 1, wherein two or more regions of a nucleic acid molecule are detected.

4. The method of claim 1, wherein the two or more nucleic acid molecules or the two or more regions of the nucleic acid molecule are in close proximity to each other due to direct interactions.

5. The method of claim 1, wherein the two or more nucleic acid molecules or the two or more regions of the nucleic acid molecule are in close proximity to each other due to indirect interactions in a complex of molecules.

6. The method of claim 5, wherein the two or more nucleic acid molecules or the two or more regions of the nucleic acid molecule are in close proximity to each other due to indirect interactions in a nucleic acid-protein complex.

7. The method of claim 1, wherein the nucleic acids are double-stranded.

8. The method of claim 1, wherein the nucleic acids are single-stranded.

9. The method of claim 1, wherein the nucleic acids are DNA.

10. The method of claim 1, wherein the nucleic acids are RNA.

11. The method of claim 1, wherein the method comprises analyzing each compartment for the presence or absence of the two or more nucleic acid molecules or two or more regions of the nucleic acid molecule.

12. The method of claim 1, wherein the detecting step comprises amplifying the nucleic acid molecules or the regions of the nucleic acid molecule.

13. The method of claim 12, wherein the amplifying step comprises PCR, quantitative PCR, or real-time PCR.

14. The method of claim 1, wherein the detecting step comprises nucleotide sequencing the nucleic acid molecules or the regions of the nucleic acid molecule.

15. The method of claim 1, wherein the detecting step comprises detecting one or more agents that hybridize to the nucleic acid molecules or to the regions of the nucleic acid molecule.

16. The method of claim 15, wherein the one or more agents are fluorophores.

17. The method of claim 1, wherein the method comprises:
contacting the nucleic acids with at least two agents, wherein the first agent hybridizes to a first nucleic acid molecule or a first region of a nucleic acid molecule and wherein the second agent hybridizes to a second nucleic acid molecule or a second region of a nucleic acid molecule; and
detecting the presence of the first agent and the second agent; thereby determining that the two or more nucleic acid molecules or the two or more regions of the nucleic acid molecule in the sample are in close proximity to each other.

18. The method of claim 17, wherein the first agent and the second agent combine to produce a signal that is not generated in the absence of the first agent, the second agent, or both.

19. The method of claim 1, wherein the providing step comprises isolating the nucleic acids from the sample.

20. The method of claim 19, wherein the isolating does not substantially disrupt direct or indirect interactions between nucleic acid molecules or between regions of nucleic acid molecules in the sample.

21. The method of claim 19, wherein the isolated nucleic acids are resuspended in a solution.

22. The method of claim 21, wherein the isolated nucleic acids are resuspended in a solution comprising one or more reagents for detecting the nucleic acid molecules or the regions of the nucleic acid molecule.

23. The method of claim 22, wherein the one or more reagents are oligonucleotide probes.

24. The method of claim 1, wherein the sample is an extract from an animal, plant, bacterial, or viral source.

25. The method of claim 1, wherein the sample comprises one or more cells.

26. The method of claim 25, wherein the providing step comprises disrupting or dissolving a cell membrane of the one or more cells.

27. The method of claim 25, wherein the providing step comprises permeabilizing a cell membrane of the one or more cells.

28. The method of claim 1, wherein the sample comprises an isolated cell nucleus.

29. The method of claim 1, wherein the providing step comprises nucleic acid shearing or nuclease digestion of the nucleic acids.

30. The method of claim 1, wherein the providing step comprises purifying the nucleic acids from other components in the sample.

31. The method of claim 1, wherein the compartmentalizing step comprises diluting the mixture.

32. The method of claim 31, wherein the diluting comprises sequentially diluting the mixture to generate a plurality of dilutions and compartmentalizing each of the plurality of dilutions into a plurality of compartments.

33. The method of claim 1, wherein the compartmentalizing step comprises partitioning the mixture into droplets.

34. The method of claim 33, wherein the droplets are surrounded by an immiscible carrier fluid.

35. The method of claim 1, wherein the compartmentalizing step comprises partitioning the mixture into microcapsules.

36. The method of claim 1, wherein the providing step comprises providing the mixture of nucleic acids under conditions such that proteins remain bound to the nucleic acid molecules or regions of the nucleic acid molecule in the mixture.
